(19) United States
(12) Patent Application Publication
(10) Pub. No.: US 2018/0346413 A1
(43) Pub. Date: Dec. 6, 2018

(54) PROCESS FOR PREPARING ISOCYANATE COMPOUND

(71) Applicant: BASF SE, Ludwigshafen (DE)

(72) Inventors: Thomas SCHAUB, Neustadt (DE); Robert BAUMANN, Mannheim (DE); Rocco PACIELLO, Bad Duerkheim (DE); Nicolas GERMAIN, Heidelberg (DE)

(73) Assignee: BASF SE, Ludwigshafen (DE)

(21) Appl. No.: 15/571,274

(22) PCT Filed: May 4, 2016
(86) PCT No.: PCT/EP2016/059955

§ 371 Date: Nov. 2, 2017

(30) Foreign Application Priority Data
May 6, 2015 (EP) 15166596.5

(51) Int. Cl. C07C 263/04; C07C 269/04

(52) U.S. Cl. CPC C07C 263/04; C07C 269/04

(57) ABSTRACT

A process for preparing an isocyanate compound is provided. The process includes a step of reacting an amine compound A having at least one primary amino group with CO₂ and an organotin compound S having at least one radical OR³ attached to the tin atom of the organotin compound to convert at least one of the primary amino groups in the amine compound A into a carbamate group to obtain a carbamate compound C; a step of cleaving the carbamate groups in the obtained carbamate compound C to form the isocyanate compound and an alcohol R³OH, without separation of the tin compounds; and a step of obtaining the isocyanate compound. The radical R³ is a C-bound organic radical of 1-30 carbon atoms with 1, 2, or 3 carbon atoms optionally replaced by oxygen or nitrogen.

The present invention relates to a process for preparing isocyanate compounds, which process comprises the reaction of at least an amine compound with CO₂ and cleavage of the thus obtained carbamate compound.

Isocyanates are an industrial important class of compounds, mainly as monomers for the production of polyurethanes. On the common synthetic pathway, isocyanates are produced on the industrial scale by the reaction of amines with phosgene (see: C, Six, F, Richter, Isocyanates, Organic in Ullmann's Encyclopedia of Industrial Chemistry, 2012, 63-82). The main disadvantages of this process are the high safety precautions necessary due to the handling of the extremely toxic phosgene and the recycling of the hydrochloric acid formed as a by-product during the synthesis.

Therefore, alternative pathways to the use of phosgene for the isocyanate synthesis are becoming important (see: O. Kreye, H. Mutlu, M. A. R. Meier, Green Chemistry, 2013, 15, 1431-1455).

Principally, CO₂ may serve an alternative to phosgene in the synthesis of isocyanates from amines. The direct synthesis of isocyanates from CO₂ and an amine, however, is not possible due to the instability of the carbamic acid formed as an intermediate (see: C, Six, F, Richter, Isocyanates, Organic in Ullmann's Encyclopedia of Industrial Chemistry, 2012, 63-82) and the unfavorable thermodynamic equilibrium of this reaction. In order to drive the equilibrium into the right direction, a base, e.g. tertiary amines, such as triethylamine, and stoichiometric amounts of harsh drying agents like POCl₃ or P₄O₁₀ are required. The use of harsh drying agents and the formation of ammonium salts as by-products are undesirable.

So far, neither simple recycling protocols for the formed salts nor protocols for the regeneration of the drying agent have been described (see: T. E. Waldmann, W. D. McGhee, J. Chem. Soc., Chem. Commun., 1994, 957-958; D.C. Dean, M. A. Wallace, T. M. Marks, D. G. Melillo, Tetrahedron Lett.,1997, 38, 919-922).

It is known to use dialkyl carbonates as a CO₂ building block for preparing carbamates and/or isocyanates from amines.

WO 2011/051314 describes the preparation of isocyanates from amines in a multi stage reaction sequence involving the formation of dialkyl carbonates in a two-step process from CO₂ with glycols formed as by-product. In a first step, an alkylene oxide is reacted with CO₂ to obtain a cyclic carbonate. The cyclic carbonate is then reacted with a monoalcohol to the glycol (stoichiometric by-product) and a non-cyclic dialkylcarbonate. The carbonate is then reacted with an amine to the carbamate, which can be thermally cleaved to the desired isocyanate and the monoalcohol. The alcohol can be recycled in the formation step of dialkylcarbonate. This process requires a stoichiometric amount of an alkylene oxide. Furthermore, the process produces a stoichiometric amount of a glycol as by-product. Therefore, this process requires chemical sites, where alkylene oxides (mainly ethylene oxide or propylene oxide) are available and therefore limits the flexibility to build-up such a process.

JP2013107909 describes a process for preparing isocyanate, wherein a diaryl carbonate and an amine compound are reacted in the presence of an aromatic hydroxy compound as a solvent. The obtained aryl carbamic acid is subjected to a thermal decomposition reaction to obtain isocyanate.

G. Zhu et al., Ind. Eng. Chem. Res., 2013, 52, 4450-4454 relates to a kinetic study for the cleavage of methyl N-phenyl carbamate to phenyl isocyanate. The decomposition of the carbamate was obtained under high pressure using chlorobenzene as solvent without any catalyst. The results of these studies show that the presence of a catalyst during the decomposition of the carbamate compound promotes side reactions and decreases the yield of the isocyanate compound.

It is an object of the present invention to provide a process for the synthesis of isocyanate compounds which allows for avoiding the use of phosgene. The process should be more efficient than the known processes requiring less process steps. The process should be feasible for the synthesis of both aliphatic and cycloaliphatic isocycanate compounds as well as for aromatic isocyanate compounds.

Surprisingly, it has now been found that these objectives are achieved by a process comprising the steps of reacting an amine compound having at least one primary amino group with CO₂ and an organotin compound defined below, to convert at least one of the primary amine groups into a carbamate group followed by cleavage of the carbamate group to form the isocyanate compound and an alcohol.

Accordingly, the present invention relates to a process for the preparation of an isocyanate compound comprising the steps of:
a) Reacting an amine compound A having at least one primary amine group with CO₂ and an organotin compound S having at least one radical OR³ attached to the tin atom of the organotin compound, wherein R³ is a C-bound organic radical having from 1 to 30 carbon atoms, wherein 1, 2 or 3 carbon atoms may be replaced by oxygen or nitrogen, to convert at least one of the primary amine groups in the amine compound A into a carbamate group, thereby obtaining a carbamate compound C;
b) cleavage of the carbamate groups in the carbamate compound C obtained in step a) to form the isocyanate compound and an alcohol R³OH, without separation of the tin compounds formed in step a);
c) obtaining the isocyanate compound from the reaction mixture of step b).

The process according to the invention is associated with several advantages. Primarily, the process is more efficiently by requiring less process steps than to the known processes involving the use of CO₂. Moreover, the process of the invention avoids the formation of stoichiometric by-products. A further advantage is the avoidance of phosgene. Moreover, the process allows for the synthesis aromatic isocyanate compounds. Furthermore, at least a part of the alcohol obtained by the cleavage of the carbamate compound can be used to regenerate the organotin compound or organotin residue which is formed though the reaction of the organotin compound S with the amine A and CO₂. The unreacted reactants, the alcohol and the regenerated organotin compound S can thus be recycled into the reaction, thereby providing a highly economical process.

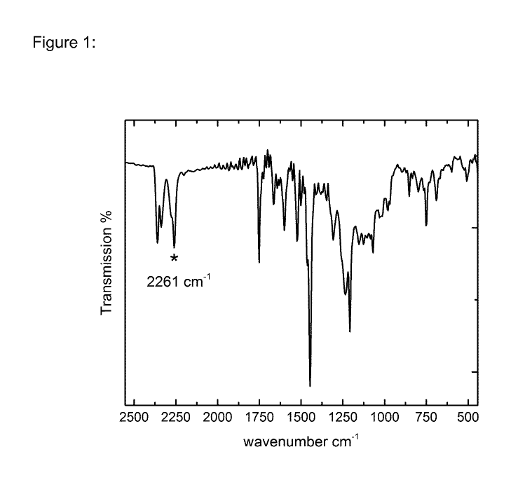

FIG. 1 shows an IR spectra of the crude mixture of example 3-1 after 2 hours.

In the context of the invention, the generic terms used hereinafter, such as halogen, alkyl, haloalkyl, alkoxy, cycloalkanediyl, aryl and arylene, are defined as follows:

In the context of the generic terms, the prefix Cₓ-C_{y} denotes the number of possible carbon atoms a generically defined radical may have.

The term “halogen” denotes in each case fluorine, bromine, chlorine or iodine, especially fluorine, chlorine or bromine.

The term “C₁-C₄-alkyl” denotes a linear or branched alkyl radical comprising from 1 to 4 carbon atoms, for example CH₃, C₂H₅, n-propyl, CH(CH₃)₂, n-butyl, CH(CH₃)—C₂H₅, CH₂—CH(CH₃)₂ and C(CH₃)₃.

The term “C₁-C₆-alkyl” denotes a linear or branched alkyl radical comprising from 1 to 6 carbon atoms, for example C₁-C₄-alkyl as mentioned above, and also, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably methyl, ethyl, n-propyl, 1-methylethyl, n-butyl, 1,1-dimethylethyl, n-pentyl or n-hexyl.

The term “C₁-C₄-haloalkyl”, as used herein and in the haloalkyl units of C₁-C₄-halo-alkoxy, describes straight-chain or branched alkyl groups having from 1 to 4 carbon atoms, where some or all of the hydrogen atoms of these groups have been replaced by halogen atoms. Examples thereof are chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 3,3,3-trifluoroprop-1-yl, 1,1,1-trifluoroprop-2-yl, 3,3,3-trichloroprop-1-yl, heptafluoroiso-propyl, 1-chlorobutyl, 2-chlorobutyl, 3-chlorobutyl, 4-chlorobutyl, 1-fluorobutyl, 2-fluoro-butyl, 3-fluorobutyl, 4-fluorobutyl and the like.

The term “alkoxy” denotes straight-chain or branched saturated alkyl groups comprising from 1 to 6 (C₁-C₆-alkoxy) or 1 to 4 (C₁-C₄-alkoxy) carbon atoms which are bound via an oxygen atom to the remainder of the molecule, such as methoxy, ethoxy, n-propoxy, 1-methylethoxy (isopropoxy), n-butyloxy, 1-methylpropoxy (sec-butyloxy), 2-methylpropoxy (isobutyloxy) and 1,1-dimethylethoxy (tert-butyloxy).

The term “C₁-C₁₈-alkyl” denotes a linear or branched alkyl radical comprising from 1 to 18 carbon atoms. Examples are, as well as the radicals specified for C₁-C₄-alkyl or C₁-C₆-alkyl, pentyl, hexyl, heptyl, octyl, 2-ethylhexyl, nonyl, decyl, undecyl, dodecyl, 2-propylheptyl, 3-butyloctyl, tridecanyl, tetradecanyl, pentadecanyl, hexadecany, heptadecanyl, octadecanyl and positional isomers thereof.

The term “cycloalkyl” denotes monocyclic saturated hydrocarbon groups having 3 to 16 (C₃-C₁₆-cycloalkyl) or 3 to 12 (C₃-C₁₂-cycloalkyl) carbon ring members, such as cyclobutyl, cyclopentyl, cyclohexyl, cycloheptyl, cyclooctyl, cyclononyl, cyclodecanyl, cyclo undecanyl, cyclododecanyl, dodecanyl, cyclotridecanyl, cyclotetradecanyl, cyclopentadecanyl and cyclohexadecanyl.

The term “cycloalkyl-C₁-C₆-alkyl” denotes cycloalkyl radicals which are bound via a C₁-C₆-alkyl group to the remainder of the molecule examples are cyclopropylmethyl (CH₂-cyclopropyl), cyclobutylmethyl, cyclopentylmethyl, cyclohexylmethyl, 1-cyclopropylethyl (CH(CH₃)-cyclopropyl), 1-cyclobutylethyl, 1-cyclopentylethyl, 1-cyclohexylethyl, 2-cyclopropylethyl (CH₂CH₂-cyclopropyl), 2-cyclobutylethyl, 2-cyclopentylethyl or 2-cyclohexylethyl.

The term “cycloalkanediyl” refers to a divalent saturated cyclic hydrocarbon radical which has from 3 to 12 carbon atoms, such as cyclopropane-1,2-diyl, cyclobutane-1,3-diyl, cyclopentane-1,2-diyl, cyclohexane-1,4-diyl, cycloheptane-1,3-diyl.

The term “aryl” denotes carbocyclic aromatic radicals having from 6 to 14 carbon atoms. Examples thereof comprise phenyl, naphthyl, fluorenyl, azulenyl, anthracenyl and phenanthrenyl. Aryl is preferably phenyl or naphthyl, and especially phenyl.

The term “aryl-C₁-C₆-alkyl” denotes aryl radicals which are bound via a C₁-C₆-alkyl group to the remainder of the molecule. Examples thereof are benzyl, 2-phenylethyl (phenethyl) and the like.

The term “(C₁-C₈-alkoxy)-C₁-C₈-alkyl” denotes alkoxy radicals which are bound via a C₁-C₆-alkyl group to the remainder molecule. Examples are methoxymethyl, ethoxymethyl, n-propoxymethyl, butoxymethyl, 1-methoxyethyl, 1-ethoxyethyl, 1-(n-propoxy)ethyl, 1-butoxyethyl, 2-methoxyethyl, 2-ethoxyethyl, 2-(n-propoxy)ethyl, 2-butoxyethyl, 2-methoxypropyl, 2-ethoxypropyl, 2-(n-propoxy)propyl, 2-butoxypropyl.

The term “C₆-C₁₄-arylene” denotes divalent aromatic radicals having from 6 to 14 or 6 to 10 carbon atoms, such as benzene-1,2-diyl, benzene-1,3-diyl, benzene-1,4-diyl or naphthalene-1,2-diyl.

Step a)

In step a) of the process of the present invention, an amine compound A having at least one primary amine group is reacted in the presence of an organotin compound S having at least one radical OR³ attached to the tin atom of the organotin compound, wherein R³ is a C-bound organic radical having from 1 to 30 carbon atoms, wherein 1, 2 or 3 carbon atoms may be replaced by oxygen or nitrogen with CO₂.

The organotin compound S has at least one radical OR³ attached to the tin atom of the organotin compound S, wherein R³ is a C-bound organic radical having 1 to 30 carbon atoms, wherein 1, 2 or 3 carbon atoms may be replaced by oxygen or nitrogen and does not have protic functional groups.

In particular, R³ is selected from C₁-C₁₈-alkyl, C₃-C₁₆-cycloalkyl, C₃-C₁₆-cycloalkyl-C₁-C₄-alkyl, C₆-C₁₄-aryl and C₆-C₁₄-aryl-C₁-C₄-alkyl, wherein the 5 aforementioned radicals are unsubstituted or substituted with 1, 2, 3, 4 or 5 substituents independently selected from halogen, C₁-C₆-alkyl and C₁-C₄-alkoxy.

Examples of suitable groups R³ include but are not limited to methyl, ethyl, 1-propyl, 2-propyl, 1-butyl, 2-butyl, tert-butyl, 1-(2-methyl)propyl, 2-(2-methyl)propyl, 1-pentyl, 1 -(2-methyl)pentyl, 1 -hexyl, 1 -(2-ethyl)hexyl, 1 -heptyl, 1 -2(propyl)heptyl, 1 -octyl, 1-nonyl, 1-decyl, 1-undecyl, 1-dodecyl, trifluoromethyl, 2,2,2-trifluoroethyl, pentafluoroethyl, methoxymethyl, ethoxymethyl, 2-methoxyethyl, 2-ethoxyethyl, 3-methoxypropyl, 3-ethoxypropyl, 2-methoxypropyl, 2-ethoxypropyl, adamantly, cyclopentyl, methylcyclopentyl, cyclohexyl, methycyclohexyl, cycloheptyl, cyclooctyl, norbonyl, phenyl, naphthyl, tolyl, xylyl, chlorophenyl and anisyl.

More particularly, R³ is C₁-C₆-alkyl which is unsubstituted or carries 1, 2, 3, 4 or 5 substituents selected from F and C₁-C₄-alkoxy, such as methoxy or ethoxy.

Especially R³ is C₁-C₆-alkyl, C₁-C₄-alkyl, which is substituted by 1 to 4 fluorine atoms and C₁-C₄-alkoxy-C₁-C₄-alkyl.

In a particular preferred embodiment the organotin compound S has the formula (I)

R¹R²Sn(OR³)₂ (I)

wherein R¹ and R² are identical or different and selected from C-bound organic radicals having from 1 to 30 carbon atoms, and R³ has one of the meanings as defined above.
In particular, R¹ and R² are identical or different and selected from C₁-C₁₈-alkyl, C₁-C₈-alkoxy-C₁-C₈-alkyl, C₃-C₁₆-cycloalkyl, C₃-C₁₆-cycloalkyl-C₁-C₄-alkyl, C₆-C₁₄-aryl and C₆-C₁₄-aryl-C₁-C₄-alkyl, wherein the 6 aforementioned radicals are unsubstituted or substituted with 1, 2, 3 or 4 substituents independently selected from halogen, C₁-C₆-alkyl and C₁-C₄-alkoxy, and
especially, R¹ and R² are C₁-C₆-alkyl, such as methyl, ethyl, 1-propyl, 2-propyl, 1-butyl, 2-butyl, tert-butyl, 1-(2-methyl)propyl, 2-(2-methyl)propyl, 1-pentyl, 1-(2-methyl)pentyl, 1-hexyl.

In the context of formula (I), R³ has in particular one of the aforementioned particular, more particular or special meanings. Especially, R¹ and R² are C₁-C₆-alkyl, such as methyl, ethyl, 1-propyl, 2-propyl, 1-butyl, 2-butyl, tert-butyl, 1-(2-methyl)propyl, 2-(2-methyl)propyl, 1-pentyl, 1-(2-methyl)pentyl, 1-hexyl, and R³ is C₁-C₆-alkyl, C₁-C₄-alkyl, which is substituted by 1 to 4 fluorine atoms and C₁-C₄-alkoxy-C₁-C₄-alkyl. Particular examples of organotin compound of the formula (I) include but are not limited to dimethyl dimethoxy stannan, diethyldimethoxy stannan, dimethyl diethoxy stannan, di-n-butyldimethoxy stannan, di-n-butyldibutoxy stannan, di-n-butyl-di-(2-methoxy-ethoxy) stannan, dimethyl-(2-methoxy-ethoxy) stannan, dimethyl-di-(2,2,2-trifluoro-ethoxy) stannan, di-n-butyl-di- (2,2,2-trifluoro-ethoxy) stannan, di-n-butyldiisobutoxy stannan and di-methyldiisobutoxy stannan.

The terms organotin by-product, organotin residue and organotin compound formed in step a) are used synonymously. They are different from the organotin compound S by the fact that they lack a residue OR³ as they are formed in the reaction of organotin compound S, the amine A and CO₂. It is assumed that the organotin by-product or organotin residue comprises Sn—OH, Sn═O, and/or Sn—O—Sn subunits. The organotin by-product or organotin residue can be monomeric, dimeric, oligomeric, polymeric, cyclic or mixtures thereof tin-containing compounds.

The organotin compound formed in step a) may be monomeric, dimeric or oligomeric or higher polymers. It is assumed that the organotin compound formed in step a) may have one of the following structures A to E or may be a mixture thereof. Examples for a monomeric organotin compound are the compounds defined by formulae A and B:

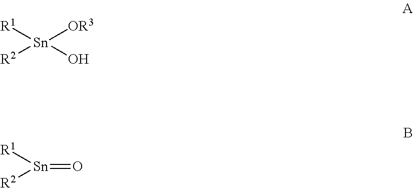

wherein R¹, R² and R³ have one of the meanings defined herein.

Examples for a dimeric, oligomeric and polymeric are the compounds of formulae C to E:

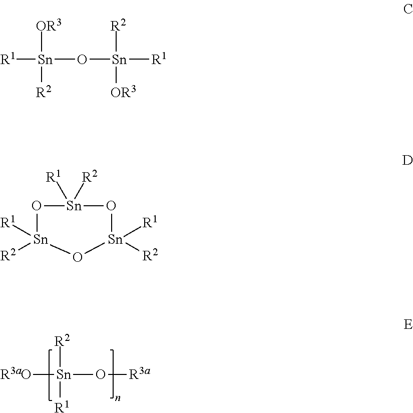

wherein R¹, R² have one of the meanings as defined above, Ria is hydrogen or has one of the meanings of R³ as defined herein and n is an integer that reflects the degree of polymerization of the polymeric structure.

The amine compound A comprises at least one primary amine group NH₂. In particular, the amine compound A has one or two primary amine group NH₂.

Among the amine compounds further particular groups (1) of embodiments relate to the amine compounds of formula (II)

H₂NR⁴ (II),

wherein
R⁴ is an organic radical having from 1 to 30 carbon atoms, wherein 1, 2 or 3 carbon atoms may be replaced by oxygen or nitrogen;
in particular, R⁴ is selected from C₁-C₁₂-alkyl, C₃-C₁₂-cycloalkyl, C₃-C₁₂-cycloalkyl-C₁-C₄-alkyl, C₆-C₁₄-aryl and C₆-C₁₄-aryl-C₁-C₄-alkyl, wherein the 5 aforementioned radicals are unsubstituted or substituted with 1, 2, 3 or 4 substituents independently selected from halogen, C₁-C₆-alkyl and C₁-C₄-alkoxy;
more particularly, R⁴ is selected from C₁-C₁₂-alkyl, C₃-C₉-cycloalkyl, C₃-C₉-cycloalkyl-C₁-C₄-alkyl, C₆-C₁₀-aryl and C₆-C₁₀-aryl-C₁-C₄-alkyl, wherein the 5 aforementioned radicals are unsubstituted or substituted with 1, 2, 3 or 4 substituents independently selected from halogen, C₁-C₂-alkyl and C₁-C₂-alkoxy.

Examples of suitable groups R⁴ include but are not limited to methyl, ethyl, 1-propyl, 2-propyl, 1-butyl, 2-butyl, tert-butyl, 1-(2-methyl)propyl, c-(2-methyl)propyl, 1-pentyl, 1-(2-methyl)pentyl, 1-hexyl, 1-(2-ethyl)hexyl, 1-heptyl, 1-2(propyl)heptyl, 1-octyl, 1-nonyl, 1-decyl, 1-undecyl, 1-dodecyl, adamantly, cyclopentyl, methylcyclopentyl, cyclohexyl, methycyclohexyl, cycloheptyl, cyclooctyl, norbonyl, phenyl, naphthyl, tolyl, xylyl, chlorophenyl and anisyl;

Particular examples of the compounds of formula (II) include 1-aminohexane, 1-amino-2-ethylhexane, aniline, 2-aminotoluene, 3-aminotoluene, 4-aminotoluene, and aminoxylenes, and mixtures thereof, i.e. R⁴ is 1-hexyl, 1-(2-ethyl)hexyl, phenyl, 2-tolyl, 3-tolyl, 4-tolyl 1,2-dimethyl-3-phenyl, 1,3-dimethyl-4-phenyl, or 1,4-dimethyl-2-phenyl.

Further particular groups (2) of embodiments relate to the diamine compounds of formula (III)

H₂NXNH₂ (III),

wherein
X is a bivalent organic radical having from 2 to 30 carbon atoms, wherein 1, 2 or 3 carbon atoms may be replaced by oxygen or nitrogen;
preferably, X is selected from C₂-C₁₂-alkanediyl, C₃-C₁₂-cycloalkanediyl, C₆-C₁₄-arylene, wherein C₃-C₁₂-cycloalkanediyl and C₆-C₁₄-arylene are unsubstituted or with 1, 2, 3 or 4 substituents independently selected from halogen, C₁-C₆-alkyl and C₁-C₄-alkoxy,
   L-R^{x}, R^{x}-L′-R^{x}, wherein
   L is selected from C₁-C₁₂-alkanediyl, C₃-C₁₂-cycloalkanediyl and C₆-C₁₄-arylene,
   L′ is selected from O, S, SO₂, C₁-C₁₂-alkanediyl, C₃-C₁₂-cycloalkanediyl and C₆-C₁₄-arylene,
R^{x}, R^{x}′ independently of each other are selected from C₃-C₁₂-cycloalkandiyl and C₆-C₁₄-arylene, wherein the 2 aforementioned radicals are unsubstituted or substituted with 1, 2, 3 or 4 substituents independently selected from halogen, C₁-C₆-alkyl and C₁-C₄-alkoxy,
particularly, X is selected from C₂-C₈-alkanediyl, C₃-C₆-cycloalkanediyl, phenylene, such as 1,4-phenylene, wherein C₃-C₆-cycloalkanediyl and phenyene are unsubstituted or with 1, 2, 3 or 4 substituents independently selected from halogen, C₁-C₆-alkyl and C₁-C₄-alkoxy,
   L-R^{x}, R^{x}-L′-R^{x}, wherein
   L is selected from C₁-C₈-alkanediyl, C₃-C₆-cycloalkanediyl and phenylene, such as 1,4-phenylene,
   L′ is selected from O, S, SO₂, C₁-C₁₂-alkanediyl, C₃-C₁₂-cycloalkanediyl and phenylene, such as 1,4-phenylene;
R^{x}, R^{x}′ independently of each other are selected from C₃-C₆-cycloalknediyl and phenylene, such as 1,4-phenylene, wherein the 2 aforementioned radicals are unsubstituted or substituted with 1, 2, 3 or 4 substituents independently selected from fluorine, C₁-C₄-alkyl and C₁-C₄-alkoxy;
especially, X is selected from C₂-C₈-alkanediyl, C₃-C₆-cycloalkanediyl, and phenylene, wherein C₃-C₆-cycloalkanediyl and phenylene are unsubstituted or substituted with 1, 2, 3 or 4 substituents independently selected from fluorine, C₁-C₄-alkyl and C₁-C₄-alkoxy,
more especially, X is selected from C₂-C₈-alkanediyl, C₃-C₆-cycloalkanediyl, and phenylene, wherein C₃-C₆-cycloalkanediyl and phenylene are unsubstituted or substituted with 1 or 2 substituents which is independently selected from C₁-C₄-alkyl.

Particular examples of the compounds of formula (III) include 1,6-diaminohexane, 1,2-, 1,3- and 1,4-diaminobenzol, 2,3-diaminotoluene, 2,4-diaminotoluene, 2,5-diaminotoluene, 2,6-diaminotoluene, 4,4′-diaminodiphenylmethane, bis(4-aminocyclohexyl)methane, and isophoronediamine, i.e. X is selected from hexamethylene, phenylene, 2,3-toluene-diyl, 2,4-toluene-diyl, 2,5-toluene-diyl, 2,6-toluene-diyl, diphenylmethane-4,4′-diyl, bis(cyclohexanyl)methane-4,4′-diyl and isophoronediyl. Particular preference is given to 1,6-diaminohexane and diaminotoluene, including the mixture of its isomers such as 2,3-diaminotoluene, 2,4-diaminotoluene, 2,5-diaminotoluene, 2,6-diaminotoluene, 3,4-diaminotoluene, 3,5-diaminotoluene and mixtures thereof. A particular preference is also given to 4,4′-diaminodiphenylmethane.

In step a), at least one of the primary amine groups of the amine compound A is concerted into a carbamate group, thereby obtaining a carbamate compound C. The carbamate compound is characterized by having a radical of the formula

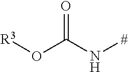

where R³O stems from the OR³ group of the organotin compound and # indicates the binding site to the remainder of the amine compound.

In a preferred embodiment, a compound of formula (II) is used as the amine compound. Hence, the resulting carbamate compound C will be a compound of formula (IV)

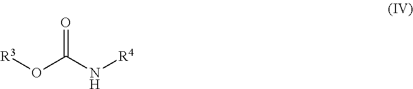

wherein R³ and R⁴ has one of the meanings as defined above in context with formula

(I) and (II).

In another preferred embodiment, a compound of formula (III) is used as the amine compound. Hence, the resulting carbamate compound C has the structure of formula (V).

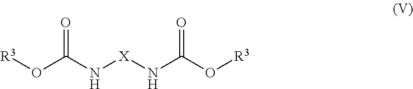

wherein R³ and X have one of the meanings as defined above;

The organotin compound S used in step a) according to the process of the invention is generally employed in an amount from 0.9 to 10 mol per mol of primary amine groups in the amine compound A, in particular in an amount of 1 to 5 mol pro mol of primary amine groups in the amine compound A.

The reaction, performed in step a) according to the process of the invention is usually carried out in the liquid phase, i.e. the reactants, except for CO₂, i.e. the organotin compound and the amine compound and optional solvent, are present in the liquid state under reaction conditions.

The reaction, performed in step a) according to the process of the invention may be carried out in bulk or in an organic solvent, which generally will be an aprotic organic solvent. In this context, the term “in bulk” is understood by a skilled person that the reactants, i.e. the organotin compound and the amine compound amount at least 95% of the content of the reactor, except for CO₂ and optional gas phase.

Suitable aprotic organic solvents are in principle those which are chemically inert with regard to the reactants, intermediates and products. The aprotic organic solvents include but are not limited to
aromatic hydrocarbons having 6 to 14 carbon atoms, preferably 6 to 10 carbon atoms,
halogenated aromatic hydrocarbons having 6 to 14 carbon atoms, preferably 6 to 10 carbon atoms,
alkylated aromatic hydrocarbons having 6 to 14 aromatic carbon atoms, preferably 6 to 10 aromatic carbon atoms, and 1 to 10 aliphatic carbon atoms, preferably 1 to 4 aliphatic carbon atoms,
alkanes and cycloalkanes having 3 to 20 carbon atoms, preferably 4 to 18 carbon atoms, most preferably 5 to 16 carbon atoms,
chlorinated and/or brominated alkanes having 1 to 6 carbon atoms, preferably 1 to 4 carbon atoms, most preferably 1 to 3 carbon atoms,
linear or branched or cyclic ethers having 2 to 12 carbon atoms,
dialkyl formamides having 3 to 9 carbon atoms, preferably dialkyl formamides dialkyl 3 to 7 carbon atoms,
dialkyl sulfoxides dialkyl 2 to 8 carbon atoms,
nitrile having 1 to 4 carbon atoms,

and mixtures thereof.

The aprotic organic solvent is preferably selected from
alkanes and cycloalkanes, having 5 to 16 carbon atoms such as pentane, hexane, heptane, n-octane, isooctane, 2-ethylhexane, cyclohexane, cycloheptane, methylcyclohexane and higher alkanes, such as dodecanes, tetradecanes, hexadecanes etc.,
aromatic hydrocarbons having 6 to 10 carbon atoms, including optionally chlorinated aromatic hydroxcarbons, such as benzene, toluene, xylene, o-xylene, m-xylene, p-xylene,chlorobenzene, dicholorobenzenes and trichlorobenzes,
chlorinated alkanes having 1 to 4 carbon atoms such as dichloromethane or dichloroethane,
linear or branched or ethers having 2 to 8 carbon, such as dimethylether, diethylether, di-tert-butylether, di-n-butylether, tetrahydrofuran, 2-methyltetrahydrofuran,
dimethylformamide,
dimethylsulfoxide,
acetonitrile and propionitril,

and mixtures thereof.

The aprotic organic solvent is especially selected from
alkanes and cycloalkanes, having 5 to 16 carbon atoms such as pentane, hexane, heptane, n-octane, isooctane, 2-ethylhexane, cyclohexane, cycloheptane, methylcyclohexane and higher alkanes, such as dodecanes, tetradecanes, hexadecanes etc.,
aromatic hydrocarbons having 6 to 10 carbon atoms, including optionally chlorinated aromatic hydroxcarbons, such as benzene, toluene, xylene, o-xylene, m-xylene, p-xylene, chlorobenzene, dicholorobenzenes and trichlorobenzes and
chlorinated alkanes having 1 to 4 carbon atoms such as dichloromethane or dichloroethane,

If the reaction is performed in a solvent, the concentration of reactants, except for CO₂, is usually in the range from 1 to 90% by weight, in particular from 2 to 80% by weight, and especially from 5 to 70% by weight, based on the total weight of solvent and reactants, except for CO₂.

It is preferred to perform the reaction of step a) under conditions, where protic impurities such as water are essentially absent, i.e. the concentration of such impurities in the mixture of reactants is less than 1000 ppm by weight. Preferably, the CO₂ also contains less than 1000 ppm by weight of protic impurities such as water.

In step a) of the process according to the invention CO₂ is reacted with the amine compound A. For this, CO₂ is usually introduced into a mixture of the amine compound A and the organotin compound S and optionally an aprotic solvent. For this, CO₂ can be used in gaseous, liquid or supercritical state. It is also possible to use CO₂ comprising gas mixtures available on the industrial scale. Besides CO₂ the gas mixture may contain one or more inert gases, such as nitrogen or noble gases, such as argon. Preferably, the reaction is performed under conditions, such that the CO₂ partial pressure in the reactor is generally from 0.1 to 500 bar, preferably from 1 to 200 bar, in particular from 10 to 150 bar. Preferably, the reaction of step a) is performed at an absolute pressure from 0.1 to 500 bar, preferably from 1 to 200 bar, in particular from 10 to 150 bar.

Step a) of the process according to the invention is usually carried out at a temperature from 20 to 300° C., in particular from 30 to 250° C. and especially from 50 to 200° C. or from 100 to 180° C., depending from the reactivity of the amine and the partial pressure of CO₂ in the reaction vessel.

The reaction of step a) can be carried out in customary devices and/or reactors known to the person skilled in the art for liquid-gas reactions. Suitable standard reactors for the base exchange are indicated, for example, in K. D. Henkel, “Reactor Types and Their industrial Applications”, in Ullmann's Encyclopedia of Industrial Chemistry, 2005, Wiley-VCH Verlag GmbH&Co. KGaA, DOI: 10.1002/14356007.b04_087. Examples of standard reactors, include but are not limited to stirred tank reactors, including cascades of stirred tank reactors, tube reactors and fixed bed reactors.

Step a) of the process according to the invention can be carried out batchwise or continuously.

In the case of batch operation, the reactor is charged with the organotin compound S, the amine compound A and optionally a solvent. CO₂ is then introduced to the desired pressure and the reaction vessel is heated to desired temperature. After the reaction is complete, the reactor is generally depressurized. The reaction time is usually from 1 minute to 20 hours, in particular from 5 minutes to 2 hours.

In the case of the continuous mode of operation, the organotin compound S, CO₂ and the amine compound A and optionally a solvent are introduced continuously into the reactor. Accordingly, the product is continuously discharged from the reactor, so that the average liquid level in the reactor remains constant. Suitable reaction vessels for continuous operating step a) include cascades of stirred tank reactors and tubular reactor. If step a) is performed in continuous mode, the average residence time of the reactants in the reactor is generally from 1 minute to 10 hours, in particular from 5 minutes to 2 hours.

Preferably, step a) of the process according to the invention is carried out continuously.

Step a) may be carried out until the total amount of the amine compound in the reaction mixture has been consumed. Frequently, step a) will be carried out until conversion of the amine compound introduced into the reactor of at least 30%, in particular at least 50% has been achieved.

Step b)

In step b) of the process according to the invention the carbamate groups in the carbamate compound C formed in step a) are cleaved to form the isocyanate compound and an alcohol R³OH, wherein R³ has one of the meanings as defined herein.

In addition to the carbamate compound C, the reaction mixture obtained in step a) comprises the organotin by-products formed though the reaction in step a). According to the invention, these organotin by-products are not separated from the carbamate compound C obtained in step a). Rather, the reaction of step b) is performed in the presence of these organotin by-products.

Steps a) and b) can be performed as separate, subsequent steps. Depending on the reaction conditions of step a), however, a part of the carbamate formed in step a) may already been cleaved to the isocyanate compound and the alcohol R³OH. It may also be possible to perform steps a) and b) as a single step, i.e. by choosing reaction conditions, where the intermediate carbamate compound is cleaved to the isocyanate compound.

If a solvent is used in step a), it can be advantageous to remove the solvent by distillation to obtain the pure carbamate and organotin by product. It can be also advantageous to remove unreacted starting materials, hence the non-reacted amine A and optionally the non-reacted organotin compound S. Generally, the removal of the solvents or starting materials is carried out by distillation, preferably under reduced pressure.

The cleavage of the carbamate groups in the carbamate compound C in step b) can be carried out in the liquid phase or in the gas phase.

Preferably, the concentration of non-reacted amine is reduced to less than 10% by weight, based on the reaction mixture which is reacted in step b), in particular the concentration of non-reacted amine is reduced to less than 1% by weight, based on the reaction mixture which is reacted in step b).

Alternatively, the reaction of step b) can be carried in the presence of an organic solvent. Suitably organic solvents are aprotic organic solvents as defined above for step a), in particular those solvents, which are mentioned as preferred.

Preferably, step b) is performed at a temperature in the range from 50 to 400° C., in particular from 100° C. to 350° C., especially from 120 to 240° C. The reaction temperature of step b) may be the same temperature applied in step a). It is also possible to perform step b) at a temperature, which is higher than the temperature of step a), e.g. at least 10 K higher than the reaction temperature of step a).

The reaction in step b) is preferably carried out at an absolute pressure of below 1 bar. Preferably, the total pressure of from 0.00001 to <1 bar, in particular, the absolute pressure is preferably from 0.0001 to <1 bar. The reaction of step b) may be performed at the same pressure of step a). Preferably, the reaction of step b) is performed at an absolute pressure, which is lower than the absolute pressure during step a).

The cleavage of step b) can be performed in any suitable reaction device mentioned in context of step a). It is also possible to perform step b) as a distillation, in particular, if the organotin compound has a volatility which is lower than the volatility of the isocyanate formed. In this case, the alcohol R³OH and the isocyanate compound will be obtained in the distillate.

It may also be possible to start the cleavage of step b) at a temperature or pressure, where no components will distil off and then to raise the temperature and/or to reduce the pressure to distil off the alcohol R³OH and the isocyanate compound.

The cleavage of the carbamate compound C results in an isocyanate compound and an alcohol R³OH, wherein R³ has one of the meanings as defined herein. It is apparent that by performing steps a) and b) the primary amino group(s) of the amino compounds A are converted into isocyanate groups.

Hence, using an amino compound of the formula (II) will result in the isocyanate compound of formula (VI)

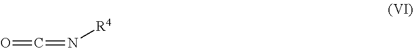

while using an amino compound of the formula (III) will result in the isocyanate compound has the structure of formula (VII)

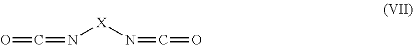

wherein R⁴ and X are as defined for formulae (II) and (III), respectively.

Step c

In step c), the isocyanate of step c) is obtained from the reaction mixture formed in step b).

Steps b) and c) may be performed as separate steps or as a single step. Preferably, the products obtained in step b) are separated from the reaction mixture by distillation.

As mentioned above for step b), this distillation may be performed in a manner such that the isocyanate is distilled off from the reaction mixture during the cleavage of the carbamate compound, thereby combining steps b) and c). In this case the heavies will contain the organotin compound and high boiling by products of the reaction. If the organotin compound has a high volatility, the organotin compound and the alcohol may be distilled off first and then the isocyanate is distilled of from high boiling by products.

In step c) of the process according to the invention, the products, i.e. the isocyanate compound and the alcohol R³OH, are preferably separated rapidly from each other in order to prevent unwanted side reactions or to prevent a high conversion respectively. A rapid separation can be achieved e.g. by performing a fractionating distillation, thereby separating the alcohol R³OH and the isocyanate compound. It is also possible to remove the alcohol R³OH under reduce pressure without condensation.

Thus, the step c) is preferably carried out in a distillation unit to separate the alcohol R³OH immediately from the isocyanate compound. The distillation unit to be used generally comprises random packing elements, ordered packings and/or bubble cap trays.

Depending on the substituents R³ of the alcohol and R⁴ or X of the isocyanate compound, either the isocyanate compound or the alcohol R³OH is the lower boiling compound. If the isocyanate compound is the lower boiling compound, the isocyanate compound is separated from the alcohol and the organotin by-products/residue. If the alcohol R³OH is the lower boiling compound, the alcohol R³OH is separated from the isocyanate compound and the organotin by-product/residue. Frequently, the alcohol R³OH will have a boiling point, which is sufficiently lower than the boiling point of the isocyanate compound, which allows for an effective fractionation.

In general, the temperature at the bottom of the distillation column is at least 50° C., preferably at least 150° C. In particular, the temperature is ≤400° C., especially ≤300° C.

The pressure is generally at least 0.00001 bar, preferably at least 0.001 bar and preferably ≤1 bar, in particular less than 0.2 bar.

If the isocyanate compound is the higher boiling compound, the isocyanate compound remains with the organotin by-products in the low boiling fraction. According to the characteristic of the organotin by-products and the substituents R¹ and R² as defined above of the organotin by-products and the substituents of R⁴ or X as defined above of the isocyanate compound the organotin by-product/residue can be separated as a higher boiling residue or a lower boiling residue.

Frequently, the isocyanate compound has a higher vapour pressure than the organotin by-products.

The process according to the invention may comprise an additional step d), wherein an alcohol R³OH, wherein R³ has one of the meanings as defined above, is reacted with the organotin by-products/residues in order to regenerate the organotin compound S.

Preferably, at least a part of the alcohol R³OH, which is obtained in step b), in particular at least 50% of the alcohol R³OH formed in step b) is used to regenerate the organotin compound/residue formed in step a) in order to obtain the organotin compound S which may then be recycled into step a).

The regeneration step d) of the organotin by-product/residue is preferably carried out in the liquid phase but it may also be carried out in the gas phase. Preferably it is carried out at a temperature from 50 to 400° C. Preferably, step d) is carried out at a temperature of at least 100° C., and preferably at a temperature of at most 350° C.

Preferably, step d) is carried out at an absolute pressure in the range from 0.00001 to 1 bar. Preferably, the absolute pressure in step d) is from 0.0001 to 1 bar.

For the regeneration step d), the water is preferably removed continuously from the reaction mixture to obtain a high conversion.

The regeneration step d) can be carried out using the bulk of the organotin compounds/residues and the alcohol R³OH. Alternatively, it is possible to add a solvent to the reaction mixture, especially to remove water by continuous azeotropic distillation. Suitably solvents are aprotic organic solvent having a mixing gap with water. Useful solvents include:
alkanes and cycloalkanes, having 5 to 16 carbon atoms such as pentane, hexane, heptane, n-octane, isooctane, 2-ethylhexane, cyclohexane, cycloheptane, methylcyclohexane and higher alkanes, such as dodecanes, tetradecanes, hexadecanes etc., and
aromatic hydrocarbons having 6 to 10 carbon atoms, including optionally chlorinated aromatic hydroxcarbons, such as benzene, toluene, xylene, o-xylene, m-xylene, p-xylene, chlorobenzene, dichlorobezene and trichlorobzene.

The regeneration step of the organotin by-product/residue is preferably carried out in a distillation unit to separate the water immediately from the organotin compound S by azeotropic distillation. In general, the temperature at the bottom of the distillation column is at least 50° C., preferably from 50° C. to 400° C., in particular from 150° C. to 300° C. The distillation is preferably carried out at a pressure in the range from 0.00001 bar to 10 bar, in particular at a pressure in the range from 0.001 bar to10 bar.

The invention is illustrated in detail by the examples below.

Analytic:

Gas chromatograph (GC): Agilent Technologies 6890N Network GC System HPLC: HPLC Chiralpka IB using a mixture of hexane/isopropanol, wherein the ratio of hexane and isopropanol is:

from 0 to 5 min: 5% isopropanol, 95% hexane,

from 5 to10 min: 15% isopropanol, 85% hexane

from 1 to 25 min: 25% isopropanol, 75% hexane

from 25 min: 50% isopropanol, 50% hexane.

¹H-NMR spectrometer:

IR spectrometer:

Commercial carbamate, Methyl N-phenylcarbamate (>98.0%): TCI

Dimethyltin(IV) dichloride (96%): Sigma Aldrich

2,4-diaminotoluene (>98%): TCI

Dimethyltin(IV) oxide (96%): Sigma Aldrich

4,4′-methylenedianliline:

1. Preparation of the Tin Compounds

### EXAMPLE 1-1

### Synthesis of Dimethyltin(IV)Dimethoxide

In a glovebox a Schlenk tube was charged with sodium methoxide (1.79 g; 33.15 mmol). Dry methanol (100 mL) was added and the obtained solution was cooled to −40° C. (dry-ice bath). To this mixture, dimethyltin(IV) dichloride (3.64 g; 16.57 mmol) was added in portions within 20 minutes. The reaction mixture was allowed to warm up to room temperature within 5 hours. After this, sodium chloride was removed by filtration of the crude mixture through a Celite path. Methanol was then distillated off under 1 atm. The desired compound was eventually recrystallized in pentane at −40° C. to give a crystalline colorless solid (1.19 g; 29%).

### EXAMPLE 1-2

### Synthesis of Dimethyltin(IV)Dimethoxide

In a glovebox a 200 mL Schlenk flask was charged with sodium methoxide (1.79 g; 33.15 mmol). Under an atmosphere of argon dry methanol (70 mL) was added to the flask and the obtained solution was cooled to 0° C. (water-ice bath). To this mixture, dimethyltin(IV) dichloride (3.64 g; 16.57 mmol) was added in portions within 20 minutes. The reaction mixture was allowed to warm up to room temperature within 13 hours. The resulting mixture was transferred into a round-bottom flask (250 mL) surmounted by a distillation apparatus. Methanol was distilled off (˜95% of the overall volume). The resulting colorless oil was transferred into the glovebox and 100 mL dry pentane was added. Insoluble material was filtered off over cellite. The desired compound (colorless crystals; 72%; 2.53 g) was obtained by crystallization from the pentane solution by storing it for 24 h at −40° C.

The synthesis of dimethyltin(IV) bis(2,2,2-trifluoroethanol) and dibutyltin(IV) bis(2,2,2-trifluoroethanol) have been prepared similarly to the example 1-1 or 1-2 mentioned above.

¹H NMR (400 MHz, [D₈]Toluene): δ=3.35 (s, 6H; MeO), 0.30 ppm (t, ³J(H—H)=3.6 Hz, 6H; CH₃);

¹³C NMR (100 MHz, [D₈]Toluene): δ=50.9, −1.7 ppm

¹¹⁹Sn NMR (298 K; 111.82 MHz; [D₈]Toluene): δ=133.1 ppm

2. Preparation of the Carbamate Compounds:

### EXAMPLE 2-1

### Synthesis of 2,4-toluene Dimethylcarbamate

In a glovebox, a stainless 60 mL steel Premex® autoclave was charged with dibutyltin(IV) dimethoxide (1.68 g; 6 mmol), 2,4-diaminotoluene (0.122 g.; 1 mmol) and pentane (10 mL). The autoclave was sealed and, outside the glovebox, pressurized with 50 bar of carbon dioxide. The mixture was stirred and after 10 minutes an equilibrium of the CO₂ uptake was achieved. Then the mixture was heated to 135° C. After 3 hours, the autoclave was cooled by a water ice-bath. After 30 minutes, the pressure was released. The autoclave was then unsealed. The crude mixture was filtered and the yellow solid was washed with cold pentane (three times). Analytically pure 2,4-toluene dimethylcarbamate was obtained (77% ,185 mg). The tin species remains in the organic solvent and can be regenerated from this phase and reused in carbamation reaction.

¹H NMR (200 MHz, [D₂]Dichloromethane): δ=7.78 (s, 1H; Hₐᵣ), 7.16-7.03 (m, 2H; Hₐᵣ), 6.79 (bs, 1H; NH), 6.48 (bs, 1H; NH), 3.73 (s, 3H; NHCO₂CH₃), 3.71 (s, NHCO₂CH₃, 3H), 2.16 ppm (s, 3H; ArCH₃);

¹³C NMR (100 MHz, [D₂]Dichloromethane): δ=154.5, 154.3, 137.2, 136.8, 131.0 (3C), 52.7, 52.5, 17.1 ppm;

Elementar Analysis: calcd (%) for C₁₁H₁₄N₂O₄ (238.1): C 55.46, H 5.92, N 11.76; found:

C 54.66, H 6.64, N 11.19.

MS (El): m/z(%): 256.43 [M+H₂O])

The cleavage of 2,4-toluene dimethylcarbamate to 2,4-toluene diisocyanate can be carried out in analogy to the example 3-1 to 3-3 mentioned below.

### EXAMPLE 2-2

### Synthesis of Methylene diphenyl-4,4′-dimethylcarbamate

In a glovebox, a stainless 60 mL steel Premex® autoclave was charged with dibutyltin(IV) dimethoxide (1.68 g; 6 mmol), 4,4′-methylenedianliline (0.204 g; 1 mmol) and pentane (10 mL). The autoclave was sealed and, outside the glovebox, pressurized with 50 bar of carbon dioxide. The mixture was stirred and after 10 minutes an equilibrium of the CO₂ uptake was achieved. Then the mixture was heated to 135° C. After 3 hours, the autoclave was cooled by a water ice-bath. After 30 minutes, the pressure was released. The autoclave was then unsealed. The crude mixture was filtered and the yellow solid was washed with cold pentane (three times). Analytically pure methylene diphenyl-4,4′-dimethylcarbamate was obtained (42% ,185 mg). The tin species remains in the organic solvent and can be regenerated from this phase and reused in the subsequent carbamation reaction.

¹H NMR (400 MHz, [D₆]dimethylsulfoxide): δ=9.52 (bs, 2H; NH), 7.35 (d, ³J(H—H)=8.4 Hz, 4H; Hₐᵣ), 7.11 (d, ³J(H—H)=8.4 Hz, 4H; Hₐᵣ), 3.79 (s, 2H; CH₂), 3.64 ppm (s, 6H; NHCO₂CH₃);

¹³C NMR (100 MHz, [D₆]dimethylsulfoxide): δ=154.0, 137.0, 135.5, 128.8, 118.4, 114.0, 51.5 ppm

Elementar Analysis: calcd (%) for C₁₇H₁₈N₂O₄ (314.1): C 64.96, H 5.77, N 8.91; found C 65.5, H 5.8, N 9.1.

MS (EI): mlz(%): 314.53 [M+H₂O])

The cleavage of methylene diphenyl-4,4′-dimethylcarbamate to diphenylmethane-4,4′-diisocyanat can be carried out in analogy to the example 3-1 to 3-3 mentioned below.

3. Preparation of the Isocyanate Compound

### EXAMPLE 3-1

### Synthesis of Ethyl N-phenylcarbamate and Phenyl Isocyanate

In a glovebox, a stainless steel Premex® autoclave was charged with dibutyltin(IV) dimethoxide (3 equiv.; 930 mg; 3 mmol), aniline (1 equiv.; 93.1 mg; 1 mmol) and 1,2,4-trichlorobenzene (10 mL). The autoclave was sealed and, outside the glovebox, pressurized with 50 bar of carbon dioxide. The mixture was stirred and after 10 minutes an equilibrium of the CO₂ uptake was achived (41 bar). Heating to the desired temperature was eventually started (pressure of 69 bar at 135° C.). After 3 hours of reaction, the autoclave was cooled down to room temperature. The pressure was released and the autoclave was unsealed and opened up in the glovebox. The crude mixture was transferred into a round-bottom flask (25 mL). Outside the glovebox, methyl N-phenyl methyl carbamate and 1,2,4-trichlorobenzene were distillated off at 60° C. (oil bath temperature) under 1.10⁻³ mbar leaving bis(dimethylmethoxytin(IV)) oxide which was converted to dimethyltin(IV) dimethoxide. The flask containing the solution of methyl N-phenylcarbamate (MPC) in 1,2,4-trichlorobenzene was surmounted by a condenser and the solution was heated to 230° C. for 5 hours. The solution was then cooled down to room temperature and absolute ethanol (5 mL) was added. The resulting mixture was heated again to 100° C. for 3 hours. The crude mixture was coiled down to room temperature. An aliquot was taken for HPLC analysis. After this, absolute ethanol (5 mL) was added. The resulting mixture was heated again to 100° C. for 3 hours. After cooling down the crude to room temperature, a sample was taken for HPLC analysis.

Before the addition of ethanol, phenyl isocyanate was identified.

Similar results were obtained when decalin was used as a solvent instead.

HPLC data were collected from HPLC Chiralpka IB using a mixture of hexane/isopropanol, wherein the ration of hexane and isopropanol is as defined above. Commercial phenyl isocyanate (with the UV fingerprint) were used as referenced for HPLC analysis: t=12.90; area [%]=100.

The H PLC-spectra for an aliquot of the crude mixture after 1 hour shows the following peaks:

aniline (identified by UV fingerprint): t=11.779, area [%]=2.295,

phenyl isocyanate: t=12.902, area [%]=25.173,

diphenylurea (identified by UV fingerprint and HPLC-MS):t=13.251, area [%]=1,705,

methyl N-phenylcarbamate: t=24.653, area [%]=70.827.

The H PLC-spectra for an aliquot of the crude mixture after 2 hour shows the following peaks:

phenyl isocyanate: t=13.014, area [%]=55.869,

methyl N-phenylcarbamate: t=25.101, area [%]=44.131.

The IR-spectra of the crude mixture after 2 hour (see FIG. 1) shows a signal at 2261 cm⁻¹, which can be assigned to characteristic signal of the phenylisocyanat (in accordance with the IR spectra of commercial phenylisocyanate as well as the values given in literature).

After quenching the isocyanate with absolute ethanol and reaction under the conditions described above (5 hours of thermolysis), the HPLC-spectra shows the following peaks:

methyl Aphenylcarbamate: t=24.918, area [%]=37.882,

ethyl N-phenylcarbamate: t=26.099, area [%]=62.118.

### EXAMPLE 3-2

### Synthesis of Methyl N-phenylcarbamate and Phenylisocyanate

In a glovebox, a 60 mL stainless-steel Premex® autoclave was charged with dimethyltin(IV)dimethoxide (211 mg; 1 mmol) and aniline (93 mg, 1 mmol). The solvent according to table 1 below was added and the autoclave was sealed. The autoclave was pressurized with carbon dioxide (50 bar) at room temperature and the mixture stirred for ten minutes. The pressure dropped to 30 bar. Afterwards, the autoclave was heated to 150° C., whereby the pressure increased to the pressure given below in table 1. After the reaction, the conversion and yield of carbamate/isocyanate was measured directly from the crude reaction mixture by gas chromatography. The gas chromatogram showed that isocyanate is formed directly out of the crude reaction mixture, obtaining the tin residue, by cleaving the carbamate under the conditions of the evaporation in the GC-oven (250° C., ambient pressure). The carbamate was cleaved into phenyl isocyanate and aniline. It was observed that the ratio of the phenyl isocyanate to aniline varied as a function of the injection temperature. The reaction can be carried in toluene, 1,2,4-trichlorobenzene, acetonitrile, dichloromethane or pentane. It was also observed that an increase of tin alkoxides equivalents improved the conversion.

A mixture of commercial carbamate (methyl N-phenylcarbamate) and dimethyltin(IV) oxide in dichloromethane was also analyzed by GC. Depending of the injection temperature, different ratios of aniline, isocyanate and carbamate were detected, whereas a single peak was observed after injection of a solution of carbamate in dichloromethane. This supports that dimethyltin(IV) oxide also forms during the reaction between aniline, carbon dioxide and diemthyltin(IV)dimethoxide and that the dimethyltin(IV) oxide is responsible for carbamate cleavage during the GC injection.

Reaction of dimethyltin(IV) bis(2,2,2-trifluoroethanol) or dibutyltin(IV) bis(2,2,2-trifluoroethanol) with aniline and carbon dioxide under the same conditions resulted in the formation of phenyl isocyanate after GC injection.

TABLE 1

| | | | | Conversion | Ratio |
|---|---|---|---|---|---|
| | | | Pressure at | Aniline [%]; | PhNCO:PhNHCOOMe; |
| | Solvent | Reaction time | 150° C. | determined by | determined by |
| Entry | [mL] | [h] | [bar] | GC-Area % | GC-Area % |
| 1 | Toluene (10) | 16 | 71 | 48 | 17:31 |
| 2 | Toluene (10) | 27 | 71 | 54 | 16:38 |
| 3 | Toluene (20) | 16 | 71 | 28 | 9:19 |
| 4 | CH₂Cl₂ (10) | 16 | 71 | 71 | 26:37 |
| 5 | Pentane (10) | 27 | 16 | 67 | 23:44 |

### EXAMPLE 3-3

### Cracking of the Crude Mixture Containing Methyl N-phenylcarbamate, Dimethyltin(IV)oxide and the Solvent:

In a glovebox, a 25 mL round-bottom flask was charged with methyl N-phenyl carbamate (1.65 g, 10 mmol) and dimethyltin(IV) oxide (1.52 g, 10 mmol). 1,2,4-trichlorobenzene (25 mL) was added and the flask was connected to a head-column. The mixture was heated at 180° C. under vacuum (5.10⁻² bar) for 16 hours. Isocyanate was detected in GC.

### EXAMPLE 3-4

### Synthesis of Methyl N-[3-(methoxycarbonylamino)-4-methyl-phenyl]-carbamate and 2,4-diisocyanato-1-methyl-benzene

In a glovebox, a 60 mL stainless-steel Premex® autoclave was charged with dimethyltin(IV)dimethoxide (422 mg; 2 mmol) and 2,4-diaminotoluene (122 mg, 1 mmol). Dry dichloromethane (10 mL) was added. The autoclave was pressurized with carbon dioxide (50 bar) at room temperature and the mixture stirred for ten minutes. The pressure dropped to 30 bar. Afterwards, the autoclave was heated to 150° C., whereby the pressure increased to 70 bar. ¹H-NMR-spectrum of the crude mixture evidenced the formation of the desired compound, the dicarbamate methyl N-[3-(methoxycarbonylamino)-4-methyl-phenyl]carbamate.

The crude mixture containing methyl N-[3-(methoxycarbonylamino)-4-methyl-phenyl]-carbamate was placed in a sublimation apparatus (cold finger filled with dry-ice/acetone) and heated under vacuum to 200° C. The 2,4-diisocyanato-1-methyl-benzene was collected and observed by ¹H-N MR-spectrum.

1. A process for preparing an isocyanate compound, the process comprising:
a) reacting an amine compound A comprising at least one primary amino group with CO₂ and an organotin compound S comprising at least one radical OR³ attached to the tin atom of the organotin compound, wherein R³ is a C-bound organic radical comprising from 1 to 30 carbon atoms, wherein 1, 2 or 3 carbon atoms are optionally replaced by oxygen or nitrogen, to convert at least one of the primary amino groups in the amine compound A into a carbamate group, thereby obtaining a carbamate compound C;
b) cleaving the carbamate groups group in the carbamate compound C obtained in a) to form the isocyanate compound and an alcohol R³OH, without separation of the tin compounds formed in a); and
c) obtaining the isocyanate compound from the reaction mixture of b).

2. The process of claim 1, wherein the organotin compound S is employed in an amount of from 0.9 to 10 mol per mol of primary amino groups in the amine compound A.

3. The process of claim 1, wherein a) is carried out in bulk or in an aprotic organic solvent.

4. The process of claim 1, wherein a) is carried out by introducing CO₂ in a reaction mixture containing the amine compound A and the organotin compound S.

5. The process of claim 1, wherein b) is carried out as a distillation of the reaction mixture obtained in a) to obtain the isocyanate compound.

6. The process of claim 1, wherein unreacted starting material of a) is removed before b) is carried out.

7. The process of claim 1, further:
adding an alcohol R³OH to the reaction mixture of b) after having obtained the isocyanate compound from the reaction mixture of b) in order to regenerate the organotin compound from the organotin compounds formed in a) or b).

8. The process of claim 7, wherein at least a part of the alcohol R³OH used to regenerate the organotin compound from the organotin compounds formed in a) or b) is the alcohol formed in b).

9. The process of claim 1, wherein the isocyanate compound has a lower vapor pressure than the organotin compounds.

10. The process of claim 1, wherein the organotin compound S is represented by formula (I)
R¹R²Sn(OR³)₂ (I)
wherein R¹ and R² are independently a C-bound organic radicals radical having from 1 to 30 carbon atoms, and R³ is as defined in claim 1.

11. The process of claim 10, wherein R¹ and R² are independently selected from the group consisting of C₁-C₁₈-alkyl, C₁-C₈-alkoxy-C₁-C₈-alkyl, C₃-C₁₆-cycloalkyl, C₃-C₁₆-cycloalkyl-C₁-C₄-alkyl, C₆-C₁₄-aryl and C₆-C₁₄-aryl-C₁-C₄-alkyl, which are unsubstituted or substituted with 1, 2, 3 or 4 substituents independently selected from the group consisting of halogen, C₁-C₆-alkyl and C₁-C₄-alkoxy.

12. The process of claim 1, wherein R³ is selected from the group consisting of C₁-C₁₈-alkyl, C₃-C₁₆-cycloalkyl, C₃-C₁₆-cycloalkyl-C₁-C₄-alkyl, C₆-C₁₄-aryl and C₆-C₁₄-aryl-C₁-C₄-alkyl, which are unsubstituted or substituted with 1, 2, 3 or 4 substituents independently selected from the group consisting of halogen, C₁-C₆-alkyl and C₁-C₄-alkoxy.

13. The process of claim 1, wherein the amine compound A comprises 1 or 2 primary NH₂ groups.

14. The process of claim 13, wherein the amine compound A is a compound of formula (II) or a compound of formula (III):
H₂NR₄ (II)
H₂NXN H₂ (III),
wherein
R⁴ is an organic radical comprising from 1 to 30 carbon atoms, wherein 1, 2 or 3 carbon atoms are optionally replaced by oxygen or nitrogen; and
X is a bivalent organic radical comprising from 2 to 30 carbon atoms, wherein 1, 2 or 3 carbon atoms are optionally replaced by oxygen or nitrogen.

15. The process of claim 14, wherein
the amine compound A is a compound of the formula (II), where
R⁴ is selected from C₁-C₁₂-alkyl, C₃-C₁₂-cycloalkyl, C₃-C₁₂-cycloalkyl-C₁-C₄-alkyl, C₆-C₁₄-aryl and C₆-C₁₄-aryl-C₁-C₄-alkyl, which are unsubstituted or substituted with 1, 2, 3 or 4 substituents independently selected from the group consisting of halogen, C₁-C₆-alkyl and C₁-C₄-alkoxy,
or
the amine compound A is a compound of formula (III), where
X is C₂-C₁₂-alkanediyl, C₃-C₁₂-cycloalkanediyl, C₆-C₁₄-arylene, L-R^{x}, or R^{x}-L′-R^{x}, wherein C₃-C₁₂-cycloalkanediyl and C₆-C₁₄-arylene are unsubstituted or substituted with 1, 2, 3 or 4 substituents independently selected from the group consisting of halogen, C₁-C₆-alkyl and C₁-C₄-alkoxy,
L is C₁-C₁₂-alkanediyl, C₃-C₁₂-cycloalkanediyl or C₆-C₁₄-arylene,
L′ is O, S, SO₂, C₁-C₁₂-alkanediyl, C₃-C₁₂-cycloalkanediyl or C₆-C₁₄-arylene,
R^{x}, R^{x}′ independently of each other are selected from C₃-C₁₂-cycloalkandiyl or C₆-C₁₄-arylene, both of which are unsubstituted or substituted with 1, 2, 3 or 4 substituents independently selected from the group consisting of halogen, C₁-C₆-alkyl and C₁-C₄-alkoxy.

16. The process of claim 14, wherein the amine compound A is 1,6-diaminohexane, a diaminotoluene, or a mixture of isomers thereof.
